(19) United States
(12) Patent Application Publication
(10) Pub. No.: US 2008/0167223 A1
(43) Pub. Date: Jul. 10, 2008

(54) Methods and Compositions for Using Tsh in the Inhibition of Tnf Activity

(75) Inventors: Mone Zaidi, Riverdale, NY (US); Etsuka Abe, Riverdale, NY (US); Terry Davies, New York, NY (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: MOUNT SINAI SCHOOL OF MEDICINE, NEW YORK, NY (US)

(21) Appl. No.: 11/547,735

(22) PCT Filed: Apr. 13, 2005
(86) PCT No.: PCT/US2005/012592

§ 371 Date: Sep. 17, 2007

Related U.S. Application Data
(60) Provisional application No. 60/562,871, filed on Apr. 16, 2004.

(51) Int. Cl. A61K 38/16; C12N 5/00; A61P 29/00

(52) U.S. Cl. 514/12; 435/375

(57) ABSTRACT

The present invention is direct to methods and compositions for inhibiting TNF activity. In particular it has been found that TSH may be used to inhibit the activity of TNF. Methods and compositions for exploiting this finding are described.

### BACKGROUND

1. Field of the Invention

The present invention is generally directed to inhibiting the inflammatory effects of TNF. More particularly, the present invention is directed to decreasing TNF activity using TSH.

2. Background of the Related Art

Tumor necrosis factor-α (TNF-α) was initially discovered as an antineoplastic cytokine and was expected to be used as an anticancer agent. However, TNF-α was subsequently identified as a proinflammatory cytokine and confirmed to be the same substance as cachectin (a cachexia inducer). Various activities were subsequently attributed to TNF-α, including stimulation of IL-1 and other cytokine production, proliferation of fibroblasts, induction of endotoxin shock, increasing ICAM-1, ICAM-2 (intercellular adhesion molecules) activity, ELAM (endothelial leukocyte adhesion molecule-1), etc. (these molecules are proteins for adhering leukocytes to endothelial cells) to accelerate the adhesion of leukocytes to endothelial cells, and for causing arthritis by stimulating bone resorption, cartilage destruction and the like. (Beutler et al., Nature, 316, 552-554, 1985; Peetre et al., J. Clin. Invest, 78, 1694-1700, 1986; Kurt-Jones et al., J. Immunol., 139, 2317-2324, 1987; Bevilacqua et al., Science, 241, 1160-1165, 1989).

TNF-α activity also is found in synovial fluid or serum of chronic rheumatoid arthritis (Saxne et al., Arthritis Rheum., 31, 1041, 1988; Chu et al., Arthritis Rheum., 34, 1125-1132 1991; Macnaul et al., J. Immunol., 145, 4154-4166 1990; Brennan et al., J. Immunol., 22, 1907-1912, 1992; Brennan et al., Brit. J. Rheum., 31, 293-298 1992). It is present in abundance in inflamed synovial tissue and exerts an important role in the pathogenesis of various autoimmune disorders (Annu. Rep. Med. Chem., 32:241-250, 1997). The sputum of patients with acute respiratory distress syndrome (ARDS) contains high concentrations of TNF-α (Millar et al., Nature, 324, 73, 1986). The activity of this cytokine also becomes elevated in viral and bacterial infections. Elevation of TNF-α also is seen in myocardial ischemia such as acute myocardial infarction (Latini et al., J. Cardiovasc. Pharmacol., 23, 1-6, 1994; Finkel, et al., Science, 257, 387-389, 1992; Pagani, et al., J. Clin. Invest., 90, 389-398, 1992). TNF-α-mediated inflammatory response also is believed to be associated with various cancers (e.g., prostate cancer) and dementias (Curr. Drug Target Inflamm Allergy 1(2) 193-200, 2002).

Thus, despite the fact that TNF-α initially was identified as an anticancer therapeutic agent, it has since been discovered that it is important to decrease the activity of this cytokine in various disorders. This has led to an extensive body of research dedicated to the identification of agents that can regulate the tissue or serum levels of TNF-α. While a number of inhibitors of TNF-α levels and activity have been reported, it is not clear whether such compounds possess the appropriate pharmacological properties to be therapeutically useful. Moreover, as many of these agents are small synthetic molecules, the physiological effects of these agents require further validation. Therefore, there is a continued need for compositions that inhibit TNF-α.

### SUMMARY OF THE INVENTION

In one aspect, the invention is directed to methods of inhibiting tumor necrosis factor (TNF) activity, in a cell that expresses TNF or a TNF receptor, by contacting the cell with a composition comprising thyroid stimulating hormone (TSH). The method may be carried out in vitro or in vivo.

Another aspect of the invention provides a method of decreasing an inflammatory response in an animal comprising administering to said animal a composition comprising TSH in an amount effective to inhibit the activity and/or expression of TNF in said animal. The TSH may be administered as a protein composition. Alternatively, the TSH is administered as an expression construct comprising a polynucleotide having a TSH-encoding nucleic acid sequence operably linked to a promoter that allows the expression of said TSH in said animal. The methods of the invention may be employed as part of a combination therapy in which a second composition is which comprises an anti-inflammatory agent administered along with the TSH. The second composition may be administered before, after or during the administration of the TSH.

The inflammatory disease being treated may be any inflammatory disease caused by an aberrant expression or activity of TNF. For example, the inflammatory disease may be caused by a viral infection.

In another aspect of the invention, methods are provided for treating any disease characterized by an elevated TNF activity and/or expression in an animal comprising administering to said animal a composition comprising TSH in an amount effective to decrease the TNF activity in said animal. Exemplary such diseases include, but are not limited to, an inflammatory disease, an autoimmune disease, destructive-bone disorder, a proliferative disorder, an infectious disease, and a degenerative disease.

Inflammatory diseases that are characterized by elevated TNF activity and/or expression are known to those of skill in the art and include, but are not limited to, rheumatological or autoimmune disease, atherosclerosis, restenosis, transplantation associated arteriopathy, psoriasis, multiple sclerosis, diabetes, inflammation-associated dementia, transplant rejection, stroke, and fever. Autoimmune diseases that are characterized by an elevated expression/activity of TNF include but are not limited to Graves Disease, Crohn's Disease, systemic scleroderma, arthritis, rheumatoid arthritis, psoriasis, psoriatic arthritis, graft vs. host disease, inflammatory bowel syndrome, systemic lupus erythromatosus, juvenile dermatomyositis, asthma, and acute pancreatitis. Dementias associated with inflammatory diseases that are treatable by the methods described herein include, but are not limited to a dementia selected from the group consisting of Alzeimer's disease, vascular dementia Parkinson's Disease. Any of the aforementioned diseases may be treated by the TSH-based therapies described herein. Combination therapies of these disorders using TSH, as part of a cocktail of agents to treat the specific disease are particularly contemplated.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1 shows TNFα, TNFRI and TNFRII expression in TSH wild type and knockout cells.

FIG. 2 shows characteristics of bone marrow cells.

FIG. 3 shows characteristics of Raw C3 cells.

### DESCRIPTION OF THE PREFERRED EMBODIMENTS

TNF-α activity is elevated in a variety of disorders and the involvement of this cytokine in such disorders has fuelled the continued pursuit for additional agents that inhibit the deleterious effects of this cytokine. In the present application, it is demonstrated that the TNF-α activity may be inhibited the administration of TSH. The present invention, therefore, is directed to methods and compositions for the treatment of any disorder in which TNF-α activity or expression is elevated. Such methods and compositions are described in further detail below.

### Methods and Compositions for Producing TSH

TSH is a pituitary glycoprotein hormone which plays a key role in regulating the function of the thyroid. Its release is stimulated by the hormone thyroid releasing hormone (TRH), which is formed in the hypothalamus and controls the formation and release of the thyroid hormone, thyroxine (T4). On the basis of a feedback control mechanism, thyroxine content of serum controls the release of TSH. The formation of thyroxine by the thyroid cells is stimulated by TSH by a procedure in which the TSH released by the pituitary binds to the TSH receptor of the thyroid cell membrane. Thus, to date, the primary, and indeed only, corroborated function of TSH is to regulate the synthesis and secretion of T4 from thyroid follicular cells. To date, no other uses for TSH have been postulated. However, commercial pharmaceutical preparations of TSH are readily available for the treatment if thyroid disorders, and as such, TSH is an agent that has proven pharmacological efficacy in humans and other animals and is not hindered by side effects and testing regimens that are required for small molecule inhibitors. An exemplary commercially available pharmaceutical preparation of TSH is Thyrogen® (Genzyme Inc., Cambridge, Mass.), a highly purified recombinant human thyroid stimulating hormone (rhTSH) developed for use in well-differentiated thyroid cancer patients who have had near-total or total thyroidectomy, and who must therefore take thyroid hormones.

Recent studies have developed a TSH receptor null mouse (Zaidi et al., J. Bone and Mineral Res., 17(1)S1-S541, abstract 1054, 2002; Abe et al., J. Bone and Mineral Res., 18(1)S1-S463, abstract 1188, 2003; Abe et al., Cell 115:151-162, 2003), which have led to speculation of additional roles for TSH. Those studies revealed direct effects of TSH on both components of skeletal remodeling, namely osteoblastic bone formation and osteoclastic bone resorption. These effects are mediated through the TSH receptor that is present on osteoclast and osteoblast precursors. Even a 50% decrease in TSH receptor expression results in profound osteoporosis (bone loss) along with localized focal osteosclerosis (localized bone formation). TSH inhibits osteoclastogenesis osteoclast survival in a dose-dependent manner. In addition, TSH attenuates the expression of markers of osteoclast differentiation, e.g., cathespin K, B3 integrin, TRAP and calcitonin receptor. Further, TSH inhibits RANK-L induced phosphorylation of Janus N-terminal kinase, accompanied by an inhibition of nuclear translocation of c-jun. Other RANK-L induced pathways, involved in osteoclast formation and survival remain unaffected, and there was no effect on the nuclear translocation of c-fos. TSH also has marked effects on osteoblast differentiation. TSH reduces expression of osteoblast differentiation markers alkaline phosphatase, bone sialoprotein, and osteocalcin and inhibits expression of osterix as well as RUNX-2. These studies collectively reveal the role of TSH as powerful negative regulator of bone remodeling that inhibits osteoclast and osteoblast formation using distinct molecular pathways.

In the present invention, additional surprising properties of TSH are revealed. More particularly, it has been discovered that TSH acts an inhibitor of TNF activity. Therefore, the present invention provides compositions for the therapeutic intervention of a variety of disorders that are characterized by and inflammatory or other response mediated by an abnormally high TNF-α activity. These methods rely on administration of a protein composition comprising TSH or alternatively may be effected by gene-therapy based methods of increasing TSH expression in a subject by providing an expression construct that comprises a TSH-encoding nucleic acid operably linked to a suitable promoter.

It is contemplated that commercial preparations, such as Thyrogen®, may be used in methods of decreasing, inhibiting or otherwise abrogating TNF activity, however, it should be understood that those skilled in the art also may be able to produce further TSH compositions for such uses. Such further compositions of TSH may include biologically active fragments, variants, mutants, and homologs of TSH. Methods of producing such fragments, variants, mutants and homologs of TSH are well known to those of skill in the art.

Like the other pituitary glycoprotein hormones, TSH is composed of two subunits, an α and a β subunit. The α-subunit is common to TSH as well as the other glycoproteins, LH, hCG and FSH. The β-subunit confers TSH specificity. The sequence of the TSH β subunit is known to those of skill in the art (see e.g., human sequence TTHUB, reproduced herein as SEQ ID NO:1, sequence for TSH β-subunit precursor). TSH β-subunit from other sources, e.g., rat (see GenBank Acc. No. NM_{—}013116) mouse (see GenBank Acc. No. NM_{—}009432), tilapia (see GenBank Acc. No. AB120769) zebrafish (see GenBank Acc. No. AY135147). Numerous other GenBank entries describe various TSH sequences. The sequence for human TSH α-subunit is given at GenBank Acc. No. TTHUAP (reproduced herein as SEQ ID NO:2) are readily available to those of skill in the art. Other sequences for TSH proteins are known to those of skill in the art. For example, additional disclosure of methods and compositions for preparing biologically active TSH are provided in U.S. Pat. No. 6,455,282 (incorporated herein by reference in its entirety), however, while that document provides that the two subunits are expressed under the control of separate promoters, it should be understood that the two subunits may be expressed under the control of a single promoter.

The entire TSH-β subunit is a protein is 138 amino acids in length (see SEQ ID NO:1. It is contemplated that fragments of this protein may be useful, including fragments of 25, 30, 35, 40, 45, 50, 55, 60, 65, 70, 75, 80, 85, 90, 95, 100, or more amino acids in length. The fragments that will be useful may be of any length from 25 amino acids in length to the full length sequence of the TSH β subunit as long as those fragments retain some activity as inhibitors of TNF activity. While it is contemplated that the entire TSH protein, including both the β and α subunits, will be most preferred in the therapeutic compositions of the invention. Nevertheless, it should be understood that any fragment of the TSH protein that retains a capacity to inhibit TNF activity is contemplated to be within the scope of the present invention.

In some embodiments, the TSH protein may be modified to enhance its uptake, circulation, and/or otherwise modified to render the protein more therapeutically effective. Thus, it may be desirable to prevent the degradation of the TSH in order to prolong the TNF inhibitory effects thereof. This may be achieved through the production of TSH variants that contain non-hydrolyzable peptide bonds, which are known in the art, along with procedures for synthesis of peptides containing such bonds. See U.S. Pat. No. 6,172,043 for a discussion of non-hydrolyzable bonds.

The TSH proteins useful in the invention can be linear, or may be circular or cyclized by natural or synthetic means. For example, disulfide bonds between cysteine residues may cyclize a peptide sequence. Bifunctional reagents can be used to provide a linkage between two or more amino acids of a peptide. Other methods for cyclization of peptides, such as those described by Anwer et al. (Int. J Pep. Protein Res. 36:392-399, 1990) and Rivera—Baeza et al. (Neuropeptides 30:327-333, 1996) are also known in the art.

Furthermore, nonpeptide analogs of the TSH proteins that provide a stabilized structure or lessened biodegradation, are also contemplated. Peptide TSH mimetic analogs can be prepared based on a TSH protein peptide structure by replacing one or more amino acid residues of the protein of interest by nonpeptide moieties. Preferably, the nonpeptide moieties permit the peptide to retain its natural confirmation, or stabilize a preferred, e.g., bioactive confirmation and an overall positive charge (Nachman et al., Regul. Pept 57:359-370, 1995).

The TSH therapeutic proteins used in the methods of the present invention may be modified in order to improve their therapeutic efficacy, and to decrease toxicity, increase circulatory time, or modify biodistribution. A strategy for improving drug viability is the utilization of water-soluble polymers (Greenwald et al., Crit Rev Therap Drug Carrier Syst. 2000;17:101-161; Kopecek et al., J Controlled Release., 74:147-158, 2001). The TSH preparations may be formulated with polyethylene glycol (PEG), an agent that has been widely used as a drug carrier, given its high degree of biocompatibility and ease of modification. Harris et al., Clin Pharmacokinet. 2001;40(7):539-51 Attachment to various drugs, proteins, and liposomes has been shown to improve residence time and decrease toxicity. (Greenwald et al., Crit Rev Therap Drug Carrier Syst. 2000;17:101-161; Zalipsky et al., Bioconjug Chem. 1997;8:111-118). PEG can be coupled to TSH through the hydroxyl groups at the ends of the chain and via other chemical methods; however, PEG itself is limited to at most two active agents per molecule. In a different approach, copolymers of PEG and amino acids are of interest as biomaterials which would retain the biocompatibility properties of PEG, but which would have the added advantage of numerous attachment points per molecule (providing greater drug loading), and which could be synthetically designed to suit a variety of applications (Nathan et al., Macromolecules. 1992;25:4476-4484; Nathan et al., . Bioconj Chem. 1993;4:54-62).

### Methods of Making and Isolating TSH Proteins

The present invention provides methods of using TSH proteins as therapeutic compositions for the treatment of a variety of inflammatory, autoimmune and other disorders mediated through an elevated TNF activity or expression. Such TSH proteins may be produced by conventional automated peptide synthesis methods, or by recombinant expression. General principles for designing and making proteins are well known to those of skill in the art.

A. Automated Solid-Phase Peptide Synthesis

The TSH protein fragments or indeed full-length TSH can be synthesized in solution or on a solid support in accordance with conventional techniques. The peptides can be prepared from a variety of synthetic or enzymatic schemes, which are well known in the art. Where short peptides are desired, such peptides are prepared using automated peptide synthesis in solution or on a solid support in accordance with conventional techniques. Various automatic synthesizers are commercially available and are used in accordance with known protocols. See, for example, Stewart and Young, Solid Phase Peptide Synthesis, 2d. ed., Pierce Chemical Co., (1984); Tam et al., J. Am. Chem. Soc., 105:6442, (1983); Merrifield, Science, 232: 341-347, (1986); Barany and Merrifield, The Peptides, Gross and Meienhofer, eds, Academic Press, New York, 1-284, (1979); Fields, (1997) Solid-Phase Peptide Synthesis. Academic Press, San Diego.); Andersson et al., Large-scale synthesis of peptides. Biopolymers (Pept. Sci.), 55, 227-250 (2000); Burgess et al., DiSSiMiL: Diverse Small Size Mini-Libraries applied to simple and rapid epitope mapping of a monoclonal antibody. J. Pept. Res., 57, 68-76, (2001); and Peptides for the New Millennium, Fields, J. P. Tam & G. Barany (Eds.), Kluwer Academic Publisher, Dordrecht. Numerous other documents teaching solid phase synthesis of peptides are known to those of skill in the art and may be used to synthesis epitope arrays from any allergen.

For example, the peptides are synthesized by solid-phase technology employing an exemplary peptide synthesizer such as a Model 433A from Applied Biosystems Inc. This instrument combines the FMOC chemistry with the HBTU activation to perform solid-phase peptide synthesis. Synthesis starts with the C-terminal amino acid. Amino acids are then added one at a time until the N-terminus is reached. Three steps are repeated each time an amino acid is added. Initially, there is deprotection of the N-terminal amino acid of the peptide bound to the resin. The second step involves activation and addition of the next amino acid and the third step involves deprotection of the new N-terminal amino acid. In between each step there are washing steps. This type of synthesizer is capable of monitoring the deprotection and coupling steps.

At the end of the synthesis, the protected peptide and the resin are collected, the peptide is then cleaved from the resin and the side-chain protection groups are removed from the peptide. Both the cleavage and deprotection reactions are typically carried out in the presence of 90% TFA, 5% thioanisole and 2.5% ethanedithiol. After the peptide is separated from the resin, e.g., by filtration through glass wool, the peptide is precipitated in the presence of MTBE (methyl t-butyl ether). Diethyl ether is used in the case of very hydrophobic peptides. The peptide is then washed with MTBE in order to remove the protection groups and to neutralize acidity. The purity of the peptide is further monitored by mass spectrometry and in some cases by amino acid analysis and sequencing.

The peptides also may be modified, and such modifications may be carried out on the synthesizer with minor modifications. For example, an amide residue may be added at the C-terminus of the peptide, and/or an acetyl group could be added to the N-terminus. Biotin, stearate and other modifications also may could also be added to the N-terminus.

The purity of any given peptide, generated through automated peptide synthesis, or through recombinant methods, is typically determined using reverse phase HPLC analysis. Chemical authenticity of each peptide is established by any method well known to those of skill in the art. In certain embodiments, the authenticity is established by mass spectrometry. Additionally, the peptides also are quantified using amino acid analysis in which microwave hydrolyses are conducted. In one aspect, such analyses use a microwave oven such as the CEM Corporation's MDS 2000 microwave oven. The peptide (approximately 2 μg protein) is contacted with e.g., 6 N HCl (Pierce Constant Boiling e.g., about 4 ml) with approximately 0.5% (volume to volume) phenol (Mallinckrodt). Prior to the hydrolysis, the samples are alternately evacuated and flushed with N₂. The protein hydrolysis is conducted using a two-stage process. During the first stage, the peptides are subjected to a reaction temperature of about 100° C. and held at that temperature for 1 minute. Immediately after this step, the temperature is increased to 150° C. and the reaction is held at that temperature for about 25 minutes. After cooling, the samples are dried and amino acid from the hydrolysed peptides samples are derivatized using 6-aminoquinolyl-N-hydroxysuccinimidyl carbamate to yield stable ureas that fluoresce at 395 nm (Waters AccQ Tag Chemistry Package). In certain aspects, the samples are analyzed by reverse phase HPLC and quantification is achieved using an enhanced integrator. Such conditions may readily be adapted for large scale production and/or for purification of other peptides.

B. Recombinant Protein Production.

As an alternative to automated peptide synthesis, recombinant DNA technology may be employed wherein a nucleotide sequence which encodes a TSH protein is inserted into an expression vector, which is then used to transtorm or transfect into an appropriate host cell. Such a transformed or transfected host cell is then cultivated under conditions suitable for expression as described herein below. Recombinant methods are especially preferred for producing longer polypeptides that comprise peptide sequences of the invention.

A variety of expression vector/host systems may be used to contain and express the peptide or protein coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below.

Expression vectors for use in prokaryotic hosts generally comprise one or more phenotypic selectable marker genes. Such genes generally encode, e.g., a protein that confers antibiotic resistance or that supplies an auxotrophic requirement. A wide variety of such vectors are readily available from commercial sources. Examples include pSPORT vectors, pGEM vectors Promega), pPROEX vectors (LTI, Bethesda, Md.), Bluescript vectors (Stratagene), pET vectors (Novagen) and pQE vectors (Qiagen). The DNA sequence encoding the given peptide substrate or fusion polypeptide is amplified by PCR and cloned into such a vector, for example, pGEX-3X (Pharmacia, Piscataway, N.J.) designed to produce a fusion protein comprising glutathione-S-transferase (GST), encoded by the vector, and a protein encoded by a DNA fragment inserted into the vector's cloning site. The primers for the PCR may be generated to include for example, an appropriate cleavage site. Treatment of the recombinant fusion protein with thrombin or factor Xa (Pharmacia, Piscataway, N.J.) is expected to cleave the fusion protein, releasing the substrate or substrate containing polypeptide from the GST portion. The pGEX-3X/TSH peptide construct is transformed into E. coli XL-1 Blue cells (Stratagene, La Jolla Calif.), and individual transformants were isolated and grown. Plasmid DNA from individual transformants is purified and partially sequenced using an automated sequencer to confirm the presence of the desired peptide or polypeptide encoding nucleic acid insert in the proper orientation. If the GST/TSH fusion protein is produced in bacteria as a soluble protein, it may be purified using the GST Purification Module (Pharmacia Biotech).

Alternatively, the DNA sequence encoding the protein may be cloned into a plasmid containing a desired promoter and, optionally, a leader sequence (see, e.g., Better et al., Science, 240:1041-43, 1988). The sequence of this construct may be confirmed by automated sequencing. The plasmid is then transformed into E. coli using standard procedures employing CaCl₂ incubation and heat shock treatment of the bacteria (Sambrook et al., supra). The transformed bacteria are grown in LB medium supplemented with carbenicillin, and production of the expressed protein is induced by growth in a suitable medium. If present, the leader sequence will effect secretion of the TSH protein and be cleaved during secretion.

The secreted recombinant protein is purified from the bacterial culture media standard protein purification techniques. Similar systems for the recombinant protein in yeast host cells are readily commercially available, e.g., the Pichia Expression System (Invitrogen, San Diego, Calif.), following the manufacturer's instructions. Another alternative recombinant production may be achieved using an insect system. Insect systems for protein expression are well known to those of skill in the art. In one such system, Autographa californica nuclear polyhedrosis virus (AcNPV) is used as a vector to express foreign genes in Spodoptera frugiperda cells or in Trichoplusia larvae. The TSH coding sequence is cloned into a nonessential region of the virus, such as the polyhedrin gene, and placed under control of the polyhedrin promoter. Successful insertion of TSH will render the polyhedrin gene inactive and produce recombinant virus lacking coat protein coat. The recombinant viruses are then used to infect S. frugiperda cells or Trichoplusia larvae in which the TSH is expressed (Smith et al., J Virol 46: 584, 1983; Engelhard E K et al., Proc Nat Acad Sci 91: 3224-7, 1994).

Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

It is preferable that the transformed cells are used for long-term, high-yield protein production and as such stable expression is desirable. Once such cells are transformed with vectors that contain selectable markers along with the desired expression cassette, the cells may be allowed to grow for 1-2 days in an enriched media before they are switched to selective media. The selectable marker is designed to confer resistance to selection and its presence allows growth and recovery of cells which successfully express the introduced sequences. Resistant clumps of stably transformed cells can be proliferated using tissue culture techniques appropriate to the cell.

A number of selection systems may be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprit- or aprt-cells, respectively. Also, anti-metabolite resistance can be used as the basis of selection for dhfr, which confers resistance to methotrexate; gpt, which confers resistance to mycophenolic acid; neo, which confers resistance to the aminoglycoside G418; als which confers resistance to chlorsulfuron; and hygro, which confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, β-glucuronidase and its substrate, GUS, and luciferase and its substrate, luciferin.

C. Expression Constructs for Recombinant Protein Production and for Therapeutic Purposes

In the recombinant production of the TSH proteins, it will be desirable to employ vectors comprising polynucleotide molecules for encoding the TSH derived proteins. Vectors also will be used in therapeutic methods that involve introducing TSH activity into an animal by supplying a gene that encodes TSH. Methods of preparing such TSH-encoding vectors, as well as producing host cells transformed with such vectors, and therapeutic compositions for gene therapy are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect gene. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will depend upon the specific host cell to be used, and is within the skill of the ordinary artisan. Methods for the construction of mammalian expression vectors are disclosed, for example, in Okayama and Berg (Mol. Cell. Biol. 3:280 (1983)); Cosman et al. (Mol. Immunol. 23:935 (1986)); Cosman et al. (Nature 312:768 (1984)); EP-A-0367566; and WO 91/18982.

The expression construct may further comprise a selectable marker that allows for the detection of the expression of a peptide or polypeptide. Usually the inclusion of a drug selection marker aids in cloning and in the selection of transformants, for example, neomycin, puromycin, hygromycin, DHFR, zeocin and histidinol. Alternatively, enzymes such as herpes simplex virus thymidine kinase (tk) (eukaryotic), β-galactosidase, luciferase, or chloramphenicol acetyltransferase (CAT) (prokaryotic) may be employed. Immunologic markers also can be employed. For example, epitope tags such as the FLAG system (IBI, New Haven, Conn.), HA and the 6xHis system (Qiagen, Chatsworth, Calif.) may be employed. Additionally, glutathione S-transferase (GST) system (Pharmacia, Piscataway, N.J.), or the maltose binding protein system DEB, Beverley, Mass.) also may be used. The selectable marker employed is not believed to be important, so long as it is capable of being expressed simultaneously with the nucleic acid encoding a gene product. Further examples of selectable markers are well known to one of skill in the art.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the peptide substrate or the fusion polypeptide. Thus, a promoter nucleotide sequence is operably linked to a given DNA sequence if the promoter nucleotide sequence directs the transcription of the sequence. Similarly, the phrase “under transcriptional control” means that the promoter is in the correct location and orientation in relation to the nucleic acid to control RNA polymerase initiation and expression of the gene. Any promoter that will drive the expression of the nucleic acid may be used. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Common promoters include, e.g., the human cytomegalovirus (CMV) immediate early gene promoter, the SV40 early promoter, the Rous sarcoma virus long terminal repeat, β-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well known properties, the level and pattern of expression of the protein of interest following transfection or transformation can be optimized. Inducible promoters also may be used.

Another regulatory element that is used in protein expression is an enhancer. These are genetic elements that increase transcription from a promoter located at a distant position on the same molecule of DNA. Where an expression construct employs a cDNA insert, one will typically desire to include a polyadenylation signal sequence to effect proper polyadenylation of the gene transcript. Any polyadenylation signal sequence recognized by cells of the selected transgenic animal species is suitable for the practice of the invention, such as human or bovine growth hormone and SV40 polyadenylation signals.

Also contemplated as an element of the expression cassette is a terminator. These elements can serve to enhance message levels and to minimize read through from the cassette into other sequences. The termination region which is employed primarily will be one selected for convenience, since termination regions for the applications such as those contemplated by the present invention appear to be relatively interchangeable. The termination region may be native with the transcriptional initiation, may be native to the DNA sequence of interest, or may be derived for another source.

One of the therapeutic embodiments contemplated herein involves administration of a composition that will increase the expression of TSH in a given cell or tissue in patient or subject. Such a subject is generally contacted with an expression construct capable of providing TSH to that cell in a functional form. It is specifically contemplated that TSH encoding genes known to those of skill in the art will be employed in human therapy, as could any of the gene sequence variants of the human TSH gene. Methods and compositions for making expression vectors and genetic elements employed therein are well known to those of skill in the art. Particularly preferred expression vectors for the delivery of the TSH are viral vectors such as adenovirus, adeno-associated virus, herpesvirus, vaccinia virus and retrovirus. Also preferred is liposomally-encapsulated expression vector.

It is now widely recognized that DNA may be introduced into a cell using a variety of viral vectors. In such embodiments, expression constructs comprising viral vectors containing the genes of interest may be adenoviral (see for example, U.S. Pat. No. 5,824,544; U.S. Pat. No. 5,707,618; U.S. Pat. No. 5,693,509; U.S. Pat. No. 5,670,488; U.S. Pat. No. 5,585,362; each incorporated herein by reference), retroviral (see for example, U.S. Pat. No. 5,888,502; U.S. Pat. No. 5,830,725; U.S. Pat. No. 5,770,414; U.S. Pat. No. 5,686,278; U.S. Pat. No. 4,861,719 each incorporated herein by reference), adeno-associated viral (see for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479 each incorporated herein by reference), an adenoviral-adenoassociated viral hybrid (see for example, U.S. Pat. No. 5,856,152 incorporated herein by reference) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688 each incorporated herein by reference) vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation of proteins and vectors as a pharmaceutically acceptable compositions is discussed below.

Various routes are contemplated for delivery. For example, systemic delivery is contemplated. In those cases where the individual being treated has a tumor or other localized condition, a variety of direct, local and regional approaches may be taken. For example, the site (e.g., tumor site) may be directly injected with the expression vector. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

D. Site-Specific Mutagenesis

Site-specific mutagenesis is another technique useful in the preparation of individual TSH proteins used in the methods of the invention. This technique employs specific mutagenesis of the underlying DNA (that encodes the amino acid sequence that is targeted for modification). The technique further provides a ready ability to prepare and test sequence variants, incorporating one or more of the foregoing considerations, by introducing one or more nucleotide sequence changes into the DNA. Site-specific mutagenesis allows the production of mutants through, the use of specific oligonucleotide sequences that encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids also are routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization (annealing) conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

Of course, the above described approach for site-directed mutagenesis is not the only method of generating potentially useful mutant peptide species and as such is not meant to be limiting. The present invention also contemplates other methods of achieving mutagenesis such as for example, treating the recombinant vectors carrying the gene of interest mutagenic agents, such as hydroxylamine, to obtain sequence variants.

E. Protein Purification

It will be desirable to purify the TSH-based proteins for use in the present invention. Protein purification techniques are well known to those of skill in the art. These techniques involve, at one level, the crude fractionation of the cellular milieu to polypeptide and non-polypeptide fractions. Having separated the peptides or polypeptides from other proteins, the polypeptides or peptides of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Particularly efficient methods of purifying peptides include fast protein liquid chromatography (FPLC) and high performance liquid chromatography (HPLC).

Certain aspects of the present invention concern the purification, and in particular embodiments, the substantial purification, of an encoded polypeptide, protein or peptide. The term “purified polypeptide, protein or peptide” as used herein, is intended to refer to a composition, isolated from other components; wherein the polypeptide, protein or peptide is purified to any degree relative to its naturally-obtainable state. A purified polypeptide, protein or peptide therefore also refers to a polypeptide, protein or peptide, free from the environment in which it may naturally occur.

Generally, “purified” will refer to a polypeptide, protein or peptide composition that has been subjected to fractionation to remove various other components, and which composition substantially retains its expressed biological activity. Where the term “substantially purified” is used, this designation will refer to a composition in which the polypeptide, protein or peptide forms the major component of the composition, such as constituting about 50%, about 60%, about 70%, about 80%, about 90%, about 95% or more of the proteins in the composition.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified polypeptide, protein or peptide.

### Methods of Treating Disorders to Inhibit TNF Activity

It is contemplated that TSH-based therapeutic compositions will be used in the treatment of a variety of disorders such as inflammatory diseases, autoimmune diseases, destructive bone, proliferative disorders, infectious diseases, and degenerative diseases.

A. Disorders Treated

Excessive TNF-α tissue levels have been implicated in mediating or exacerbating a number of diseases including: rheumatoid arthritis, rheumatoid spondylitis, osteoarthritis, gouty arthritis and other arthritic conditions, general sepsis, gram-negative sepsis, septic shock, endotoxic shock, toxic shock syndrome, adult respiratory distress syndrome (ARDS), cerebral malaria, chronic pulmonary inflammatory disease, silicosis, asbestosis, pulmonary sarcoidosis, bone resorption diseases (e.g., osteoporosis), graft vs. host reactions, allograft rejections, fever and myalgias due to bacterial or viral infections, influenza, cachexia secondary to acquired immune deficiency syndrome (AIDS), keloid formation, scar tissue formation, Crohn's disease, ulcerative colitis, pyresis, a number of “autoimmune diseases”, multiple sclerosis, autoimmune diabetes, systemic lupus erythromatosus, Hashimoto's thyroiditis, myasthenia gravis, multiple sclerosis, Guillan Barre syndrome, and glomerulonephritis. The treatment methods of the invention will be useful in treating such disorders and will produce an amelioration, decrease or other alleviation of one or more of the symptoms of such disorders.

As it has been established that TNF activity is elevated in certain disorders of the central nervous system (CNS), the TSH-based therapies also may be used in the treatment of a CNS condition or disorder. CNS conditions that can be treated include, but are not limited to, Alzheimer's Disease, Parkinson's Disease, multiple sclerosis, and amylotrophic lateral sclerosis. In one group of particularly preferred embodiments to be treated, the CNS condition or disorder to be treated is a brain tumor or other neoplasia (e.g., a CNS tumor such as a glioblastoma). Such tumors or neoplasia may be primary tumors or may be metastases.

Other neoplastic disorders also may be treated. In such embodiments, the TSH therapy will target directly to cancer cells, including cancers such as breast carcinoma, melanoma, and fibrosarcoma.

Pulmonary conditions that may be treated include lung disease such as asthma, allergies, an immune or autoimmune disorder, a microbial infection (e.g. bacterial, viral, fungal or parasitic infection). Other pulmonary disorders that may be treated include but are not limited to cystic fibrosis, asthmatic bronchitis, tuberculosis, bronchitis, bronchiectasis, laryngotracheobronchitis, bronchiolitis, emphysema, bronchial pneumonia, allergic bronchopneumonia, viral pneumonia, pertussis, diphtheria, spasmodic croup, pulmonary phthisis, encephalitis with retained secretions, pulmonary edema, cytomegaloviral pneumonia or miliary tuberculosis, drug-induced lung disease (e.g., after administration of penicillin, nitrofurantoin), neoplastic lung disease having lymphangitic spread pattern or bronchoalveolar cell carcinoma infectious or noninfectious granulomatous disease, hypersensitivity pneumonitis, histoplasmosis, tuberculosis, cryptogenic fibrosing alveolitis, hereditary pulmonary disorders, such as alveolar microlithiasis and bronchiectasis, eosinophilic granuloma, lympphangioleimyomatosis, and pulmonary alveolar proteinosis disorders. Symptoms of a pulmonary condition are symptoms associated with any of the pulmonary conditions described above. The classic symptoms associated with such pulmonary conditions may include coughing, exertional dyspnea, wheezing, chest pain and purulent sputum production. Other components of the syndrome which may accompany a pulmonary condition include hypoxia, CO₂ narcosis, hyperventilation, decreased expiration volume, and decreased lung capacity. Any of these symptoms may be monitored before and after the treatment at varying periods in order to determine the effectiveness of the treatment regiment.

TNF-α inhibition by TSH-based therapeutic compositions will be useful in the treatment of a variety of allergic, traumatic and other injurious disorders. Many of these disorders are classified as “inflammatory diseases,” and are characterized by activation of leukocytes leads to an impairment of normal physiologic function. Examples of such conditions include acute and chronic inflammation such as osteoarhritis, sepsis, asthma, chronic bronchitis, atopic dermatitis, urticaria, allergic rhinitis, allergic conjunctivitis, eosiniophilic granuloma, ulcerative colitis, reperfusion injury of the myocardium and brain, chronic glomerulonephritis, and adult respiratory distress syndrome (ARDS), immune and autoimmune disorders, rheumatoid arthitis, IBD (inflammatory bowel disease), lupus, MS, graft rejection, cirrhosis, sarcoidosis, granulomatous lesions, periodontitis/gingivitis, graft-vs.-host disease, contact dermatitis, and the like. Included among autoimmune disorders which may be treated using the present method are chronic active hepatitis, Graves' disease, insulin-dependent diabetes mellitus (type I), and Hasshimoto's thyroiditis. Included among inflammatory disorders which may be treated using the present method are inflammatory brain disease, inflammatory demyelinating disease, inflammatory vasculitis, inflammatory myopathies, osteomyelitis, Crohn's disease and interstitial cystitis. Additional examples of inflammatory diseases include myocardial diseases, infectious diseases, pulmonary diseases and graft rejection.

B. Monitoring TNF Inhibition

From the above discussion, it should be understood that the disease that may be treated by the present invention are limited only by the fact that such diseases are caused wholly or in part by an elevated TNF activity or expression.

Therefore, it should be understood that the above-listed conditions are merely an exemplary, rather than exhaustive list of the type of conditions that may be treated using TSH compositions to inhibit TNF activity therein.

As used herein, the term “inhibits TNF activity” includes its generally accepted meaning which includes prohibiting, preventing, restraining, and slowing, stopping or reversing progression, severity or a resultant symptom of a TNF activity or expression. Therefore, the term encompasses inhibition of TNF-α activity and/or decrease TNF-α levels. Such activities may be assayed, for example, by determining the release of TNF-α, and/or regulation of TNF-α protein levels and/or TNF-α activity. As such, the present method includes both medical therapeutic and/or prophylactic administration, as appropriate.

In order to investigate the TNF activity inhibiting properties of the TSH-based compositions of the invention, various assays may be used. The level of TNF-α protein in the blood or cell of a patient or a cell culture (i.e., within the cell or the cell culture media) can be determined by for example, assaying for immunospecific binding to TNF-α or to other proteins known to be produced as a result of the presence of active TNF-α. Such methods are known in the art and include, e.g., inmmunoassays which can be used such as competitive and non-competitive assay systems, western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A inmmunoassays and FACS analysis with labeled antibodies. Such assays well known in the art (see, e.g., Ausubel et al, eds, 1994, Current Protocols in Molecular Biology, Vol. 1, John Wiley & Sons, Inc., New York, which is incorporated by reference herein in its entirety).

Competitive binding assays can also be used to determine the level of TNF-α. One example of a competitive binding assay is a radioimmunoassay in which labeled proteins from cells expressing TNF-α (e.g., ³H or ¹²⁵I) are incubated with a TNF-α antibody in the presence of increasing amounts of unlabeled TNF-α, and the detection of the TNF-α antibody bound to the labeled TNF-α. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

TNF-α levels can also be assayed by activity, for example, TNF-α levels can be assayed by a cell line that is capable of detecting bioactive levels of cytokines like TNF-α or a growth factor. According to one embodiment, the level of bioactive TNF-α in a biological sample is detected by incubating a cell line genetically engineered with isopropyl-β-D-thiogalactopyranoside. The cell line is incubated with the sample to be tested and cell death in the cell line is monitored by determining the intensity of blue color which is indicative of a bioactive cytokine or growth factor in the sample tested. See also, e.g., Burns (1994) 20(1):40-44 for TNF activity assay of serum of patients.

The TNF inhibitory activity also may be tested in an in vivo setting. For example, a clinical trial may be set up in which five to fifty subjects are selected for the clinical study. The women suffer from SLE or rheumatoid arthritis. Because of the idiosyncratic and subjective nature of these disorders, the study has a placebo control group, i.e., the subjects are divided into two groups, one of which receives a TSH composition as the active agent and the other receives a placebo. Subjects in the test group receive TSH based drug preferably on a daily basis. The subjects are maintained on this therapy for 3-12 months. Accurate records are kept as to the number and severity of the symptoms in both groups and at the end of the study these results are compared. The results are compared both between members of each group and also the results for each patient are compared to the symptoms reported by each patient before the study began.

C. Administering TSH Compositions

As indicated herein throughout, individuals suffering from disorders mediated by an elevation in TNF activity and/or expression will benefit from treatment with TSH. This protein is a glycoprotein hormone and those of skill in the art will be aware of methods and compositions for administering hormones in a therapeutically effective amount. The term “therapeutically effective amount” as used herein is that amount of TSH that alleviates one or more of the symptoms of an elevated TNF activity and/or expression. Thus, the TSH compositions may reduce inflammation, reduce the levels of the mediators of inflammation that are induced by TNF, reduce pain associated with TNF-mediated response, or reduce or otherwise ameliorate other specific symptoms of the disease being treated.

The TSH composition used in the methods herein may be any TSH protein composition. Thyrogen® (Genzyme, Cambridge, Mass.) is a commercially available preparation of TSH and its preparation, formulation, and storage conditions provide exemplary guidance for the TSH based pharmaceutical compositions of the present invention. Thyrogen® is a recombinant preparation of TSH prepared in genetically modified Chinese hamster ovary cells. This powdered preparation of TSH that is reconstituted immediately prior to intramuscular injection to the buttock. Typically, the powder is reconstituted with 1.2 mL of sterile water for injection. As with Thryogen®, the sterile water for injection may be supplied with the kits and pharmaceutical compositions of the present invention. The pharmaceutical composition should preferably be stored at 2-8° C. (36-46° F.). While the TSH pharmaceutical compositions may be provided in liquid format, freeze-dried powder preparations may be desired for ease of storage and to minimize the risk of microbial contamination. In those embodiments where the pharmaceutical preparation is provided as a powder, after it is reconstituted, the preparation should be inspected visually for particulate matter or discoloration before use. Any such preparations exhibiting particulate matter or discoloration should not be used. If necessary, the reconstituted solution can be stored for up to 24 hours at a temperature between 2° C. and 8° C., while avoiding microbial contamination.

A dose of TSH may comprises 0.1 mg TSH/ml formulation administered, 0.2 mg TSH/ml formulation administered, 0.3 mg TSH/ml formulation administered, 0.4 mg TSH/ml, 0.5 mg TSH/ml formulation administered, 0.6 mg TSH/ml formulation administered, 0.7 mg TSH/ml formulation administered, 0.8 mg TSH/ml formulation administered, 0.9 mg TSH/ml formulation administered, 1.0 mg TSH/ml formulation administered, 1.1 mg TSH/ml formulation administered, 1.2 mg TSH/ml formulation administered, 1.3 mg TSH/ml formulation administered, 1.4 mg TSH/ml formulation administered, 1.5 mg TSH/ml formulation administered, 1.75 mg TSH/ml formulation administered, 2.0 mg TSH/ml formulation administered, 2.25 mg TSH/ml formulation administered, 2.5 mg TSH/ml formulation administered, 2.75 mg TSH/ml formulation administered, 3.0 mg TSH/ml formulation administered, 3.25 mg TSH/ml formulation administered, 3.5 mg TSH/ml formulation administered, 3.75. mg TSH/ml formulation administered, 4.0 mg TSH/ml formulation administered, 4.25 mg TSH/ml formulation administered, 4.5 mg TSH/ml formulation administered, 4.75 mg TSH/ml formulation administered, 5.0 mg TSH/ml formulation administered, or more of the TSH in the formulation administered. Such a dose may be administered by a single injection or multiple injections.

As a general guidance, it is noted that Thyrogen® has been administered at a concentration of 0.9 mg TSH/ml of injectable formulation. The specific activity of this preparation is between 4-12 IU/mg. Similar specific activities are contemplated for use in the methods described herein. For example, it is contemplated that the treatment methods described herein will employ a TSH formulation to provide between about 1 IU/mg TSH to about 20 IU/mg TSH to a patient in need thereof. The overall treatment methods may 1 IU TSH, 2 IU TSH, 3 IU TSH, 4 IU TSH, 5 IU TSH, 6 IU TSH, 7 IU TSH, 8 IU TSH, 9 IU TSH, 10 IU TSH, IU TSH, 12, IU TSH, IU TSH, 15 IU TSH, 16 IU TSH, 17 IU TSH, 18 IU TSH, 19 IU TSH, 20 IU TSH, 21 IU TSH, 22 IU TSH, 23 IU TSH, 24 IU TSH, 25 IU TSH, 26 IU TSH, 27 IU TSH, 28 IU TSH, 29 IU TSH, 30 IU TSH, 35 IU TSH, 40 IU TSH, 45 IU TSH, 50 IU TSH, 55 IU TSH, 60 IU TSH, 65 IU TSH, 70 IU TSH, 75 IU TSH, or more IU TSH to a subject in need thereof. Such a dose may be provided in a single administration or in multiple administrations.

The biological activity of the TSH may be determined by a cell-based bioassay in which cells expression a function TSH receptor and a cAMP-responsive element coupled to a heterologous reporter gene, e.g., luciferase, allows the measurement of TSH activity by determining the luciferase present in the bioassay. Those of skill in the art are aware of assays for measuring TSH activity, see e.g., World Health Organization (WHO) human pituitary derived TSH reference standard NIBSC 84/703 in vitro bioassay in which the amount of cAMP produced by a bovine thyroid microsomal preparation in response to TSH is measured (Rafferty and Das, Clinical Chemistry 45: 2207-2215, 1999).

Of course, it should be understood that the TSH may form part of a therapeutic regimen in which the TSH treatment is used in combination with a plurality of other therapies for the given disorder. As such, combination therapies discussed above are specifically contemplated.

### Combination Therapy

In the present application it is expressly contemplated that the TSH-based therapeutic compositions may be administered in combination with additional therapeutic agents that are described to decrease TNF activity and/or are designed to ameliorate the symptoms of the disorder being treated. Such additional therapeutic agents may include, without limitation, any drug or antigen or any drug- or antigen-loaded or drug- or antigen-encapsulated nanoparticle, microparticle, liposome, or micellar formulation capable of eliciting a therapeutic response in the condition being treated. Such agents may be encapsulated or loaded into nano- or microparticles, such as biodegradable nano- or microparticles.

The therapeutic agents used in combination with the TSH include peptides and non-peptide organic molecules. Such agents may include but are not limited to wound healing agents, antibiotics, anti-infectives, anti-oxidants, chemotherapeutic agents, anti-cancer agents, anti-inflammatory agents, and autoproliferative drugs. Therapeutic agents also include abortifacients, ace-inhibitor, α-adrenergic agonists, β-adrenergic agonists, α-adrenergic blockers, β-adrenergic blockers, adrenocortical steroids, adrenocortical suppressants, adrenocorticotrophic hormones, alcohol deterrents, aldose reductase inhibitors, aldosterone antagonists, 5-alpha reductase inhibitors, anabolics, analgesics, analgesics, analgesics, androgens, anesthetics, anesthetics, angiotensin converting enzyme inhibitors, anorexics, antacids, anthelmintics, antiacne agents, antiallergic agents, antialopecia agents, antiamebic agents, antiandrogen agents, antianginal agents, antiarrhythmic agents, antiarteriosclerotic agents, antiarthritic/antirheumatic agents, antiashsnatic agents, antibacterial agents, aminoglycosides, amphenicols, ansamycins, Flactams, lincosamides, macrolides, polypeptides, tetracyclines, antibacterial agents, 2,4-diaminopyrimidines, nitrofurans, quinolones and analogs, sulfonamides, -sulfones, antibiotics, anticholelithogenic agents, anticholesteremic agents, anticholinergic agents, anticoagulant agents, anticonvulsant agents, antidepressant agents, hydrazides/hydrazines, pyrrolidones, tetracyclics, antidiabetic agents, biguanides, hormones, sulfonylurea derivatives, antidiarrheal agents, antidiuretic agents, antidotes, antidote, antidote, antidote, antidote, antidyskinetic, antieczemnatic, antiemetic agents, antiepileptic agents, antiestrogen agents, antifibrotic agents, antiflatulent agents, antifungal agents, polyenes, allylamines, imidazoles, triazoles and antiglaucoma agents.

Other therapeutic agents include anti-viral agents, anti-fusogenic agents, blood brain barrier peptides (BBB peptides), RGD peptides, glucagon-like peptides, antigonadotropin, antigout, antihemorrhagic and antihistaminic agents; alkylmaine derivatives, aminoalkyl ethers, ethylenediamine derivatives, piperazines and tricyclics, antihypercholesterolemic, antihyperlipidemic, anthyperlipidemic and antihyperlipoproteinemic agents, aryloxyalkanoic acid derivatives, bile acid sequesterants, HMGCoA reductase inhibitors, nicotine acid derivatives, thyroid hormones/analogs, antihyperphosphatemic, antihypertensive agents, arlethanolamine derivatives, arloxypropanolamine derivatives, benzothiadiazine derivatives, n-carboxyalkyl derivatives, dihydropyridine derivatives, guanidine derivatives, hydrazines/phthalazines, imidazole derivatives, quaternary ammonium compounds, quinazolinyl piperazine derivatives, reserpine derivatives, sulfonamide derivatives, antihyperthyroid agents, antihypotensive agents, antihypothyroid agents, anti-infective agents, anti-inflammatory agents, anti-inflammatory agents, aminoarylcarboxylic acid derivatives, arylacetic acid derivatives, arylbutyric acid derivatives and arylcarboxylic acids.

Therapeutic agents also include arylpropionic acid derivatives, pyrazoles, pyrazolones, salicylic acid derivatives, thiazinecarboxamides, antileprotic, antileukemic, antilipemic, antilipidemic, antimalarial, antimanic, antimethemoglobinemic, antimigraine, antimycotic, antinauseant, antineoplastic and alkylating agents, antimetabolites, enzymes, androgens, antiadrenals, antiandrogens, antiestrogens, progestogens, adjunct, folic acid replenisher, uroprotective and antiosteporotic agents.

Therapeutic agents also include antipagetic, antiparkinsonian, antiperistaltic, antipheochromocytoma, antipneumocystis, antiprostatic hypertrophy, antiprotozoal, antiprozoal, antipruritic, antipsoriatic and antipsychotic agents, butyrophenes, phenothiazines, thioxanthenes, antipyretic, antirheumatic, antirickettsial, antiseborreheic and antiseptic/disinfectant agents, alcohols, aldehydes, dyes, guanidines, halogens/halogen compounds, mercurial compounds, nitrofurans, peroxides/permanganates, phenols, quinolines, silver compounds, antispasmodic, antisyphilitic, antithrombotic, antitubercular, antitumor, antitussive, antiulcerative, antiurolithic, antivenin, antivertigo and antiviral agents, purines/pyrimidines, anxiolytic, arylpiperazines, benzodiazepine derivatives, carbamates, astringent, benzodiazepine antagonist, beta-blocker, bronchodilator, ephedrine derivatives, calcium channel blockers, arylalkylamines, dihydropyridine, derivatives, piperazine derivatives, calcium regulators, calcium supplements, cancer chemotherapy agents, capillary protectants, carbonic anhydrase inhibitors, cardiac depressants, cardiotonic, cathartic, cation-exchange resin, cck antagonists, central stimulants, cerebral vasodilators, chelating agents, cholecystokin antagonists, choleitholytic agents, choleretic agents, cholinergic agents, cholinesterase inhibitors, cholinesterase reactivators, CNS stimulants, cognition activators, contraceptives, agents to control intraocular pressure, converting-enzyme inhibitors, coronary vasodilators, cytoprotectants, debriging agents, decongestants, dermatitis herpretiformis suppressants, diagnostic aids, digestive aids, diuretics, benthothiadiazine derivatives, organomercurials, pteridines, purines, steroids, sulfanamide derivatives, uracils, dopamine and receptor agonists.

Therapeutic agents also include dopamine receptor antagonists, ectoparasiticides, electrolyte replenisbers, emetics, enzymes, digestive agents, mucolytic agents, penicillin inactivating agents, proteolytic agents, enzyme inducers, estrogen antagonists, expectorant gastric and pancreatic secreation stimulants, gastric proton pump inhibitor, gastric secretion inhibitors, glucocorticoids, α-glucosidase inhibitors, gonad-stimulating principles, gonadotrophic hormones, gout suppressant, growth hormone inhibitor, growth hormone releasing factor, growth stimulant, hematinic, hemolytic, demostatic, heparin antagonist, hepatoprotectant, histamine h₁-receptor antagonists, histamine h₂-receptor antagonists, HMGCoA reductase inhibitor, hypnotic, hypocholesterermic and hypolipidemic agents.

Therapeutic agents also include hypotensive, immunomodulators, immunosuppressants, inotrophic agents, keratolytic agents, lactation stimulating hormone, laxative/cathargic, lipotrophic agents, local anesthetics, lupus erythematosus suppressants, major tranquilizers, mineralocorticoids, minor tranquilizers, miotic agents, monoamine oxidase inhibitors, mucolytic agents, muscle relaxants, mydriatic agents, narcotic agents; analgesics, narcotic antagonists, nasal decongestants, neuroleptic agents, neuromuscular blocking agents, neuroprotective agents, NMDA antagonists, nootropic agents, NSAID agents, opioid analgesics, oral contraceptives and ovarian hormones.

Therapeutic agents also include oxytocic agents, blood brain barrier proteins, GP-41 peptides, insulinotropic peptides parasympathomimetic agents, pediculicides, pepsin inhibitors, peripheral vasodilators, peristaltic stimulants, pigmentation agents, plasma volume expanders, potassium channel activators/openers, pressor agents, progestogen, prolactin inhibitors, prostaglandin/prostaglandin analogs, protease inhibitors, proton pump inhibitors, 5α-reductase inhibitors, replenishers/supplements, respiratory stimulants, reverse transcriptase inhibitors, scabicides, sclerosing agents, sedative/hypnotic agents, acyclic ureides, alcohols, amides, barbituric acid derivatives, benzodiazepine derivatives, bromides, carbamates, chloral derivatives, quinazolone derivatives and piperidinediones.

Therapeutic agents also include serotonin receptor agonists, serotonin receptor antagonists, serotonin uptake inhibitors, skeletal muscle relaxants, somatostatin analogs, spasmolytic agents, stool softeners, succinylcholine synergists, sympathomimetics, thrombolytics, thyroid hormone, thyroid inhibitors, thyrotrophic hormone, tocolytic, topical protectants, uricosurics, vasodilators, vasopressors, vasoprotectants, vitamin/vitamin sources, antichitic, antiscorbutic and antixerophthalmic agents, enzyme co-factors, hematopoietic, prombogenic agents and xanthene oxidase inhibitors.

In view of the above discussion, it should be understood that a preferred therapeutic agents are drugs. As used herein, the term “drug” includes, without limitation, any pharmaceutically active agent. Representative drugs include, but are not limited to, peptides or proteins, hormones, analgesics, anti-migraine agents, anti-coagulant agents, anti-emetic agents, cardiovascular agents, anti-hypertensive agents, narcotic antagonists, chelating agents, anti-anginal agents, chemotherapy agents, sedatives, anti-neoplastics, prostaglandins and, antidiuretic agents. Typical drugs include peptides, proteins or hormones such as insulin, calcitonin, calcitonin gene regulating protein, atrial natriuretic protein, colony stimulating factor, betaseron, erythropoietin (EPO), interferons such as α, β, or γ interferon, somatropin, somatotropin, somatostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2, and analogues thereof; analgesics such as fentanyl, sufentanil, butorphanol, buprenorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anti-coagulant agents such as heparin, hirudin, and analogues thereof; anti-emetic agents such as scopolamine, ondansetron, domperidone, metoclopramide, and analogues thereof; cardiovascular agents, anti-hypertensive agents and vasodilators such as diltiazem, clonidine, nifedipine, verapamil, isosorbide-5-mononitrate, organic nitrates, agents used in treatment of heart disorders, and analogues thereof; sedatives such as benzodiazepines, phenothiozines, and analogues thereof; narcotic antagonists such as naltrexone, naloxone, and analogues thereof; chelating agents such as deferoxamine, and analogues thereof; anti-diuretic agents such as desmopressin, vasopressin, and analogues thereof; anti-anginal agents such as nitroglycerine, and analogues thereof; anti-neoplastics such as 5-fluorouracil, bleomycin, and analogues thereof; prostaglandins and analogues thereof; and chemotherapy agents such as vincristine, and analogues thereof Representative drugs also include antisense oligonucleotides, genes, gene correcting hybrid oligonucleotides, ribozymes, aptameric oligonucleotides, triple-helix forming oligonucleotides, inhibitors of signal transduction pathways, tyrosine kinase inhibitors and DNA modifying agents. As used herein, the term “drug” also includes, without limitation, systems for gene delivery and gene therapeutics, including viral systems for gene delivery such as adenovirus, adeono-associated virus, retroviruses, herpes simplex virus, sindbus virus, liposomes, cationic lipids, dendrimers, and enzymes.

Other agents that could be used in the combination therapies include wound-healing agents such as e.g., integrins, cell adhesion molecules such as ICAM, ECAM, ELAM and the like, antibiotics, growth factors such as EGF, PDGF, IGF, bFGF, aFGF and KGF, fibrin, thrombin, RGD peptides and the like. Antiproliferative agents could also form part of the conjugates described herein, such compounds include antimetabolites, topoisomerase inhibitors, folic acid antagonists like methotrexate,purine antagonists like mercaptopurine, azathioprine, and pyrimidine antagonists like fluorouracil, cytarabine and the like. The conjugates may comprise antioxidants that prevent oxidative damage to tissue e.g., tocopherol derivatives (vitamin E), and free radical scavengers such as SOD, glutathione and the like.

Antibacterial agents may be used in the combination therapies contemplated herein, particularly in the therapeutic intervention of those disorders in which the TNF is elevated as a result of bacterial infection. The antibacterial agent may be from one of the major classes of antibiotics are (1) the beta-lactams, including the penicillins, cephalosporins and monobactams; (2) the aminoglycosides, e.g. gentamicin, tobramycin, netilmycin, and amikacin; (3) the tetracyclines; (4) the sulfonamides and trimethoprim; (5) the fluoroquinolones, e.g. ciprofloxacin, norfloxacin, and ofloxacin; (6) vancomycin; (7) the macrolides, which include for example, erythromycin, azithromycin, and clarithromycin; and (8) other antibiotics, e.g., the polymyxins, chloramphenicol and the lincosamides. Antibiotics accomplish their anti-bacterial effect through several mechanisms of action which can be generally grouped as follows: (1) agents acting on the bacterial cell wall such as bacitracin, the cephalosporins, cycloserine, fosfomycin, the penicillins, ristocetin, and vancomycin; (2) agents affecting the cell membrane or exerting a detergent effect, such as colistin, novobiocin and polymyxins; (3) agents affecting cellular mechanisms of replication, information transfer, and protein synthesis by their effects on ribosomes, e.g., the aminoglycosides, the tetracyclines, chloramphenicol, clindamycin, cycloheximide, fucidin, lincomycin, puromycin, rifampicin, other streptomycins, and the macrolide antibiotics such as erythromycin and oleandomycin; (4) agents affecting nucleic acid metabolism, e.g., the fluoroquinolones, actinomycin, ethambutol, 5-fluorocytosine, griseofulvin, rifamycins; and (5) drugs affecting intermediary metabolism, such as the sulfonamides, trimethoprim, and the tuberculostatic agents isoniazid and para-aminosalicylic acid. Some agents may have more than one primary mechanism of action, especially at high concentrations. In addition, secondary changes in the structure or metabolism of the bacterial cell often occur after the primary effect of the antimicrobial drug.

The therapeutic agent may be an anticancer agent. Anti-cancer agents (chemotherapeutic agents) are natural or synthetic molecules which are effective against one or more forms of cancer. This definition includes molecules which by their mechanism of action are cytotoxic (anti-cancer chemotherapeutic agents), those which stimulate the immune system (immune stimulators) and modulators of angiogenesis. The outcome in either case is the slowing of the growth of cancer cells. Numerous drugs fall into the category of chemotherapeutic agents useful in the treatment of neoplastic disease that are amenable to the embodiment of this application. Such agents derivatized with this technology can include anti-metabolites such as methotrexate (folic acid derivatives), fluoroaucil, cytarabine, mercaptopurine, thioguanine, petostatin (pyrimidine and purine analogs or inhibitors), a variety of natural products such as vincristine and vinblastine (vinca alkaloid), etoposide and teniposide, various antibiotics such as miotomycin, plicamycin, bleomycin, doxorubicin, danorubicin, dactomycin; a variety of biological response modifiers including interferon-alpha; a variety of miscellaneous agents and hormonal modulators including cisplatin, hydroxyurea, mitoxantome, procarbozine, aminogultethimide, prednisone, progestins, estrogens, antiestorgens such as tamoxifen, androgenic steroids, antiadrogenic agents such as flutamide, gonadotropin releasing hormones analogs such as leuprolide, the matrix metalloprotease inhibitors (MMPIs) as well as anti-cancer agents including Taxol (paclitaxel) and related molecules collectively termed taxoids, taxines or taxanes.

Included within the definition of “taxoids” are various modifications and attachments to the basic ring structure (taxoid nucleus) as may be shown to be efficacious for reducing cancer cell growth and which can be constructed by organic chemical techniques known to those skilled in the art.

Chemotherapeutics include podophyllotoxins and their derivatives and analogues. Another important class of chemotherapeutics useful in this invention is camptothecins. Another preferred class of chemotherapeutics useful in this invention are the anthracyclines (adriamycin and daunorubicin).

Another important class of chemotherapeutics are compounds which are drawn from the following list: Taxotere, Amonafide, Illudin S, 6-hydroxymethylacylfulvene Bryostatin 1, 26-succinylbryostatin 1, Palmitoyl Rhizoxin, DUP 941, Mitomycin B, Mitomycin C, Penclomedine, angiogenesis inhibitor compounds, Cisplatin hydrophobic complexes such as 2-hydrazino-4,5-dihydro-1H-imidazole with platinum chloride and 5-hydrazino-3,4-dihydro-2H-pyrrole with platinum chloride, vitamin A, vitamin E and its derivatives, particularly tocopherol succinate.

Other compounds useful in the invention include: 1,3-bis(2-chloroethyl)-1-nitrosurea (“carmustine” or “BCNU”), 5-fluorouracil, doxorubicin (“adriamycin”), epirubicin, aclarubicin, Bisantrene(bis(2-imidazolen-2-ylhydrazone)-9,10-anthracenedicarboxaldehyde, mitoxantrone, methotrexate, edatrexate, muramyl tripeptide, muramyl dipeptide, lipopolysaccharides, vidarabine and its 2-fluoro derivative, resveratrol, retinoic acid and retinol, carotenoids, and tamoxifen.

Other chemotherapeutic agents useful in the application of this invention include: Decarbazine, Lonidamine, Piroxantrone, Anthrapyrazoles, Etoposide, Camptothecin, 9-aminocamptothecin, 9-nitrocamptothecin, camptothecin-11 (“Irinotecan”), Topotecan, Bleomycin, the Vinca alkaloids and their analogs [Vincristine, Vinorelbine, Vindesine, Vintripol, Vinxaltine, Ancitabine], 6-aminochrysene, and Navelbine.

Other compounds useful in the application of the invention are mimetics of taxol, eleutherobins, sarcodictyins, discodermolides and epothiolones.

Other anticancer agents include anti-cancer agents such as fluoropyrimidines, pyrimidine nucleosides, purines, platinum analogs, anthracyclines/anthracenediones, podophyllotoxins, camptothecins, hormones and hormonal analogs, enzymes, proteins and antibodies, vinca alkaloids, taxanes, antihormonal agents, antifolates, antimicrotubule agents, alkylating agents (classical and non-classical), antimetabolites, antibiotics, topoisomerase inhibitors, antivirals, and miscellaneous cytotoxic agents, for example hydroxyurea, mitotane, fusion toxins, PZA, bryostatin, retinoids, butyric acid and derivatives, pentosan, fumagillin, and others. The objective of all antineoplastic drugs is to eliminate (cure) or to retard the growth and spread (remission) of cancer cells. The majority of the above listed antineoplastic agents pursue this objective by possessing primary cytotoxic activity, effecting a direct kill on the cancer cells. Other antineoplastic drugs stimulate the body's natural immunity to effect cancer cell death.

The combination therapies may involve combining TSH therapy with an additional anti-inflammatory agent. The anti-inflammatory agent used may be a steroid. A typical such steroid is methylprednisolone, a synthetic steroid that suppresses acute and chronic inflammation. In addition, it stimulates gluconeogenesis, increases catabolism of proteins and mobilization of free fatty acids. In addition, it potentiates vascular smooth muscle relaxation by beta adrenergic agonists, and may alter airway hyperactivity. It is also a potent inhibitor of the inflammatory response. Other similar steroids are known to those of skill in the art. Alternatively, the anti-inflammatory agent may be a non-steroidal anti-inflammatory agent.

### Pharmaceutical Compositions

Pharmaceutical compositions for administration according to the present invention can comprise either TSH alone as described above, or the TSH may be delivered as part of a combination therapy in which the pharmaceutical compositions also may include additional therapeutic agents for the treatment of the given disease being treated. Regardless of whether the active component of the pharmaceutical composition is a TSH-based composition alone, or whether it is part of a combination therapy regimen, each of these preparations is in some aspects provided in a pharmaceutically acceptable form optionally combined with a pharmaceutically acceptable carrier. These compositions are administered by any methods that achieve their intended purposes. Individualized amounts and regimens for the administration of the compositions for the treatment of the given disorder are determined readily by those with ordinary skill in the art using assays that are used for the diagnosis of the disorder and determining the level of effect a given therapeutic intervention produces.

It is understood that the suitable dose of a composition according to the present invention will depend upon the age, health and weight of the recipient, kind of concurrent treatment, if any, frequency of treatment, and the nature of the effect desired. However, the dosage is tailored to the individual subject, as is understood and determinable by one of skill in the art, without undue experimentation. This typically involves adjustment of a standard dose, e.g., reduction of the dose if the patient has a low body weight. As discussed in further detail above, Thyrogen® is a commercially available preparation of TSH and the clinical and investigative studies performed with that formulation provide express guidance as to the amounts and routes of administration that may be employed for the TSH-based therapies or the present invention.

The total dose of therapeutic agent may be administered in multiple doses or in a single dose. In certain embodiments, the compositions are administered alone, in other embodiments the compositions are administered in conjunction with other therapeutics directed to the disease or directed to other symptoms thereof.

In some aspects, the compositions of the invention are formulated into suitable pharmaceutical compositions, i.e., in a form appropriate for in vivo applications in the therapeutic intervention of a given disease. Generally, this will entail preparing compositions that are essentially free of pyrogens, as well as other impurities that could be harmful to humans or animals. In some aspects, the compositions are prepared for administration directly to the lung. These formulations are for oral administration via an inhalant, however, other routes of administration are contemplated (e.g. injection and the like). An inhaler device is any device useful in the administration of the inhalable medicament. Examples of inhaler devices include nebulizers, metered dose inhalers, dry powder inhalers, intermittent positive pressure breathing apparatuses, humidifiers, bubble environments, oxygen chambers, oxygen masks and artificial respirators.

One will generally desire to employ appropriate salts and buffers to render the compositions stable and allow for uptake of the compositions at the target site. Generally, the pharmaceutical compositions of the invention are provided in lyophilized form to be reconstituted prior to administration. Alternatively, the pharmaceutical compositions may be formulated into tablet form. Buffers and solutions for the reconstitution of the pharmaceutical compositions may be provided along with the pharmaceutical formulation to produce aqueous compositions of the present invention for administration. Such aqueous compositions will comprise an effective amount of each of the therapeutic agents being used, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions, its use in therapeutic compositions is contemplated. Supplementary active ingredients also are incorporated into the compositions.

Methods of formulating proteins and peptides for therapeutic administration also are known to those of skill in the art. Administration of these compositions according to the present invention will be via any common route so long as the target tissue is available via that route. Most commonly, these compositions are formulated for oral administration, such as by an inhalant. However, other conventional routes of administration, e.g., by subcutaneous, intravenous, intradernal, intramusclar, intramammary, intraperitoneal, intrathecal, intraocular, retrobulbar, intrapulmonary (e.g., term release), aerosol, sublingual, nasal, anal, vaginal, or transdermal delivery, or by surgical implantation at a particular site also is used particularly when oral administration is problematic. The treatment may consist of a single dose or a plurality of doses over a period of time.

In certain embodiments, the active compounds are prepared for administration as solutions of free base or pharmacologically acceptable salts in water suitably mixed with a surfactant, such as hydroxypropylcellulose. Dispersions also are prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. In some aspects, the carrier is a solvent or dispersion medium containing, for example, water, ethanol, polyol (for example, glycerol, propylene glycol, and liquid polyethylene glycol, and the like), suitable mixtures thereof, and vegetable oils. The proper fluidity is maintained, for example, by the use of a coating, such as lecithin, by the maintenance of the required particle size in the case of dispersion and by the use of surfactants. The prevention of the action of microorganisms is brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions is brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also are incorporated into the compositions.

In some aspects, the compositions of the present invention are formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also are derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. Unit doses of TSH include a unit dose that contains 1 IU, 2 IU, 3 IU, 4 IU, 5 IU, 6 IU, 7 IU, 8 IU, 9 IU, 10 IU, or more IU TSH per unit dose. In certain embodiment, parenteral administration of the therapeutic compounds is carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. Such formulations may influence the physical state, stability, rate of in vivo release and rate of in vivo clearance of the administered agents. Depending on the route of administration, a suitable dose is calculated according to body weight, body surface areas or organ size. The availability of animal models is particularly useful in facilitating a determination of appropriate dosages of a given therapeutic. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Typically, appropriate dosages are ascertained through the use of established assays for determining blood levels in conjunction with relevant dose response data. The final dosage regimen will be detained by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

It will be appreciated that the pharmaceutical compositions and treatment methods of the invention are useful in fields of human medicine and veterinary medicine. Thus the subject to be treated is a mammal, such as a human or other mammalian animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

The present invention also contemplated kits for use in the treatment of various disorders. Such kits include at least a first composition comprising the TSH proteins or expression constructs that encode TSH described above in a pharmaceutically acceptable carrier. Another component is a second therapeutic agent for the treatment of the disorder along with suitable container and vehicles for administrations of the therapeutic compositions. The kits may additionally comprise solutions or buffers for effecting the delivery of the first and second compositions. The kits may further comprise catheters, syringes or other delivering devices for the delivery of one or more of the compositions used in the methods of the invention. The kits may further comprise instructions containing administration protocols for the therapeutic regimens.

### EXAMPLE 1

### Exemplary Demonstration of Inhibition of TNF Activity by TSH

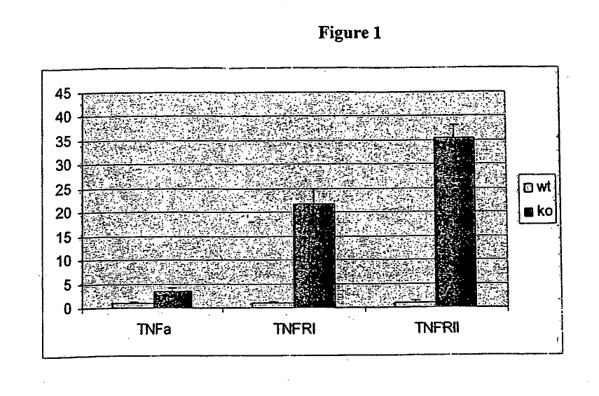

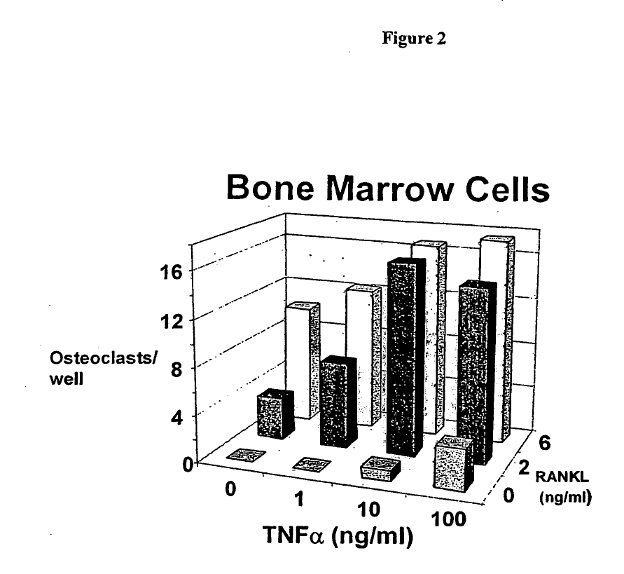

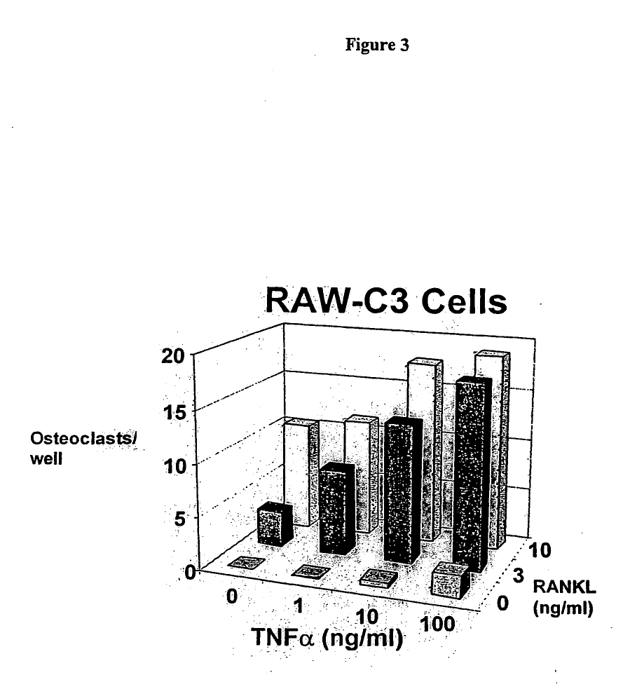

The following example is included to demonstrate the effects of TSH on TNF-α activity. It should be appreciated by those of skill in the art that this is merely an exemplification and many changes can be made in the specific embodiments which are disclosed and still obtain a like or similar result without departing from the spirit and scope of the invention. FIGS. 1 through 3 show that tumor necrosis factor activity is inhibited by contacting a cell that expresses TNF or a TNF receptor with TSH.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

What is claimed is:

1. A method of inhibiting tumor necrosis factor (TNF) activity, in a cell that expresses TNF or a TNF receptor, comprising contacting said cell with a composition comprising thyroid stimulating hormone (TSH).

2. The method of claim 1, wherein said method is an in vitro method.

3. The method of claim 1, wherein said method is an in vivo method.

4. A method of decreasing an inflammatory response in an animal comprising administering to said animal a composition comprising TSH in an amount effective to inhibit the activity and/or expression of TNF in said animal.

5. The method of claim 4, wherein said TSH is administered as a protein composition.

6. The method of claim 4, wherein said TSH is administered as an expression construct comprising a polynucleotide having a TSH-encoding nucleic acid sequence operably linked to a promoter that allows the expression of said TSH in said animal.

7. The method of claim 4, further comprising administering a second composition comprising an anti-inflammatory agent.

8. The method of claim 4, wherein said inflammatory disease is caused by a viral infection.

9. A method of treating a disease characterized by an elevated TNF activity and/or expression in an animal comprising administering to said animal a composition comprising TSH in an amount effective to decrease the TNF activity in said animal.

10. The method of claim 9, wherein said disease is selected from the group consisting of an inflammatory disease, an autoimmune disease, destructive bone disorder, a proliferative disorder, an infectious disease, and a degenerative disease.

11. The method of claim 10, wherein said inflammatory disease comprises a rheumatological or autoimmune disease, atherosclerosis, restenosis, transplantation associated arteriopathy, psoriasis, multiple sclerosis, diabetes, inflammation-associated dementia, transplant rejection, stroke, and fever.

12. The method of claim 10, wherein said autoimmune disease is selected from the group consisting of Graves Disease, Crohn's Disease, systemic scleroderma, arthritis, rheumatoid arthritis, psoriasis, psoriatic arthritis, graft vs. host disease, inflammatory bowel syndrome, systemic lupus erythromatosus, juvenile dermatomyositis, asthma, acute pancreatitis.

13. The method of claim 10, wherein said inflammatory disease is a dementia selected from the group consisting of Alzheimer's disease, vascular dementia, Parkinson's Disease.


SEQUENCE LISTING (2 sequences)

SEQ ID NO: 1 - protein, 138 aa, Homo sapiens
MTALFLMSMLFGLACGQAMSFCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 2 - protein, 116 aa, Homo sapiens
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS